(19) United States
(12) Patent Application Publication
(10) Pub. No.: US 2004/0267070 A1
(43) Pub. Date: Dec. 30, 2004

(54) Hydrotreating of Fischer-Tropsch derived feeds prior to oligomerization using an ionic liquid catalyst

(75) Inventors: David R. Johnson, Petaluma, CA (US); Thomas V. Harris, Benicia, CA (US); Michael S. Driver, San Francisco, CA (US)

Correspondence Address: CHEVRON TEXACO CORPORATION, P.O. BOX 6006, SAN RAMON, CA, 94583-0806, US

(73) Assignee: Chevron U.S.A. Inc.

(21) Appl. No.: 10/611,594

(22) Filed: Jun. 30, 2003

(51) Int. Cl. C07C 2/00

(52) U.S. Cl. 585/329

(57) ABSTRACT

A process for oligomerizing a Fischer-Tropsch derived feed containing oxygenates which comprises (a) reducing significantly the oxygenates present in the Fischer-Tropsch derived feed by contacting said feed with a hydrotreating catalyst under hydrotreating conditions in a hydrotreating zone and recovering from the hydrotreating zone a Fischer-Tropsch derived hydrotreated feed which contains a significantly reduced amount of oxygenates as compared to the Fischer-Tropsch derived feed and also a significant amount of paraffins; (b) pyrolyzing the Fischer-Tropsch derived hydrotreated feed in a thermal cracking zone under thermal cracking conditions pre-selected to crack the paraffin molecules to form olefins and collecting an olefin-enriched Fischer-Tropsch feed from the thermal cracking zone; (c) contacting the olefin-enriched Fischer-Tropsch feed with a Lewis acid ionic liquid catalyst in an oligomerization zone under oligomerization reaction conditions; and (d) recovering from the oligomerization zone a Fischer-Tropsch derived product having molecules characterized by a higher average molecular weight and increased branching as compared to the Fischer-Tropsch derived feed.

### FIELD OF THE INVENTION

This invention relates to the oligomerization of olefins in Fischer-Tropsch derived feeds by use of an ionic liquid oligomerization catalyst.

### BACKGOUND OF THE INVENTION

The economics of a Fischer-Tropsch complex has in the past only been desirable in isolated areas where it is impractical to bring the natural gas to market; however, a Fischer-Tropsch complex can benefit if the production of high-value products in the product slate, such as lubricating base oil and high quality diesel, can be increased. Fortunately, the market for lubricating base oils of high paraffinicity is continuing to grow due to the high viscosity index, oxidation stability, and low volatility relative to viscosity of these molecules. The products produced from the Fischer-Tropsch process contain a high proportion of wax which makes them ideal candidates for processing into lubricant base stocks. Accordingly, the hydrocarbon products recovered from the Fischer-Tropsch process have been proposed as feedstocks for preparing high quality lubricant base oils.

If desired, high quality diesel products also may be prepared from the syncrude recovered from the Fischer-Tropsch process. Fischer-Tropsch derived diesel typically has very low sulfur and aromatics content and an excellent cetane number. In addition, the process of the present invention makes it possible to produce diesel having low pour and cloud points which enhance the quality of the product. These qualities make Fischer-Tropsch derived diesel an excellent blending stock for upgrading lower quality petroleum-derived diesel.

Accordingly, it is desirable to be able to maximize the yields of such higher value hydrocarbon products which boil within the range of lubricating base oils and diesel. At the same time, it is desirable to minimize the yields of lower value products such as naphtha and C₄ minus products. Unfortunately, most Fischer-Tropsch processes produce lower molecular weight olefinic products within the C₃ to C₈ range. It is advantageous in a Fischer-Tropsch operation to increase the yield of higher boiling products and also increase the amount of branching in the molecules.

The average molecular weight of the hydrocarbon molecules present in the Fischer-Tropsch material may be increased by the oligomerization of olefins present in the feed. Therefore, oligomerization may be used to increase the yield of higher boiling products, such as lubricating base oils and diesel, and to lower the yield of lower boiling products, such as LPG and naphtha, from the Fischer-Tropsch process. Oligomerization also introduces desirable branching into the hydrocarbon molecule which lowers the pour point of the diesel and lubricating base oil products thereby improving the cold flow properties of the product. For those Fischer-Tropsch products intended as feed for a hydrocracking operation, a further advantage is that the branching renders the molecule easier to crack. U.S. Pat. No. 4,417,088 describes a process for oligomerizing olefins to produce molecules having desirable branching. Recently, the use of ionic liquid catalysts has been proposed for use in the oligomerization of olefins. See, for example, U.S. Pat. Nos. 5,304,615 and 5,463,158. See also European Patent Application No. 0791643 A1. U.S. Pat. No. 6,395,948 teaches that the oligomerization of alphaolefins using an ionic liquid catalyst must be conducted in the absence of an organic diluent if a polyalphaolefin having a high viscosity is desired.

Most Fischer-Tropsch derived materials as they are recovered from the Fischer-Tropsch plant will contain a certain amount of oxygenates, mostly as alcohols, but also lesser amounts of other oxygenates such as, for example, aldehydes, ketones, anhydrides, and carboxylic acids. In processes intended for upgrading the Fischer-Tropsch materials by oligomerization, the alcohols may be readily converted to olefins by dehydration, and the minor amounts of the other remaining oxygenates were not believed to be present in sufficient quantity to interfere with additional downstream processing. However, it has been found that when ionic liquid catalysts are used in the oligomerization step, even very small amounts of oxygenates will deactivate the catalyst. The present invention is intended to address this problem.

Most, but not necessarily all, of the oxygenates from the Fischer-Tropsch process will be included in the condensate fraction recovered from the unit. As used in this disclosure, the term “Fischer-Tropsch condensate” refers generally to the C₅ plus fraction which has a lower boiling point than the Fischer-Tropsch wax fraction. That is to say, the condensate represents that fraction which is normally liquid at ambient temperature. Fischer-Tropsch condensate may be obtained directly from the Fischer-Tropsch plant or produced from the Fischer-Tropsch wax by use of a wax hydrocracker. “Fischer-Tropsch wax” refers to the high boiling fraction from the Fischer-Tropsch derived syncrude and is most often a solid at room temperature.

As used in this disclosure, the words “comprises” or “comprising” are intended as an open-ended transition meaning the inclusion of the named elements, but not necessarily excluding other unnamed elements. The phrases “consists essentially of” or “consisting essentially of” are intended to mean the exclusion of other elements of any essential significance to the composition. The phrases “consisting of” or “consists of” are intended as transitions meaning the exclusion of all but the recited elements with the exception of only minor traces of impurities.

### BRIEF DESCRIPTION OF THE INVENTION

In its broadest aspect, the present invention is directed to a process for oligomerizing a Fischer-Tropsch derived feed containing oxygenates which comprises (a) reducing significantly the oxygenates present in the Fischer-Tropsch derived feed by contacting said feed with a hydrotreating catalyst under hydrotreating conditions in a hydrotreating zone and recovering from the hydrotreating zone a Fischer-Tropsch derived hydrotreated feed which contains a significantly reduced amount of oxygenates as compared to the Fischer-Tropsch derived feed and also a significant amount of paraffins; (b) pyrolyzing the Fischer-Tropsch derived hydrotreated feed in a thermal cracking zone under thermal cracking conditions pre-selected to crack the paraffin molecules to form olefins and collecting an olefin-enriched Fischer-Tropsch feed from the thermal cracking zone; (c) contacting the olefin-enriched Fischer-Tropsch feed with a Lewis acid ionic liquid catalyst in an oligomerization zone under oligomerization reaction conditions; and (d) recovering from the oligomerization zone a Fischer-Tropsch derived product having molecules characterized by a higher average molecular weight and increased branching as compared to the Fischer-Tropsch derived feed.

It has been found that oxygenates present in Fischer-Tropsch derived feeds interfere with the ability of an ionic liquid oligomerization catalyst to promote the oligomerization of the olefins, i.e., they deactivate the catalyst. Surprisingly, this interference was found to occur even when the Fischer-Tropsch feed is first subjected to a dehydration step which converts substantially all of the alcohols present into olefins. It has been discovered that even low levels of other oxygenates, such as ketones and carboxylic acids, and even low levels of residual alcohols which remain in the condensate after the dehydration step will deactivate the ionic liquid catalyst. In some cases one mole of oxygenate can deactivate one mole of catalyst. Therefore, in the present invention, a hydrotreating step is employed to reduce the amounts of the oxygenates in the Fischer-Tropsch feed intended to be sent to the oligomerization operation when an ionic liquid catalyst is employed. The entire syncrude product from the Fischer-Tropsch plant, that is to say, both the condensate and the wax fraction may be hydrotreated in the present invention. In different processing schemes within the scope of the invention, only the wax fraction or the condensate fraction may be hydrotreated. In other embodiments of the invention, only a part of one or both of the fractions may be hydrotreated. The only limitation to the material being hydrotreated being the reduction of the oxygenates in the feed being sent to the oligomerization operation to a low enough level to prevent their interference with the production of the desired product slate. In carrying out the hydrotreating operation, the hydrotreating catalyst employed and the hydrotreating conditions are selected to minimize the cracking of the hydrocarbon molecules while converting the oxygenates.

Since hydrotreating will saturate the double bonds present in the hydrocarbon molecules, following the hydrotreating step, the hydrotreated Fischer-Tropsch derived feed is pyrolyzed in a thermal cracking zone under thermal cracking conditions pre-selected to crack the paraffin molecules to create olefins prior to oligomerization. In one embodiment of the invention, the hydrotreated Fischer-Tropsch derived feed is steam cracked in a flow through reactor.

Following thermal cracking or pyrolysis, the olefin-enriched Fischer-Tropsch feed is oligomerized using an effective oligomerizing amount of a Lewis acid ionic liquid catalyst.

Following oligomerization, the Fischer-Tropsch derived product is dewaxed, if needed, to improve the cold flow properties of the products. In addition, it is usually desirable to saturate the remaining double bonds in the hydrocarbon molecules of the Fischer-Tropsch derived products. This latter operation, referred to herein as hydrofinishing, improves the UV and oxygen stability and color of the products.

The present invention also makes possible the production of higher quality lubricant base oil or a higher quantity of higher viscosity lubricant base oil than can be made by catalytic isomerization dewaxing of Fischer-Tropsch wax alone. In conventional Fischer-Tropsch operations, the amount of high viscosity lubricant base oil that can be produced by isomerization is limited by the amount of high molecular weight molecules present in the wax fraction. Oligomerization provides a method to create more high molecular weight molecules and thus more high viscosity lubricant base oil.

### BRIEF DESCRIPTION OF THE DRAWINGS

FIG. 1 is a schematic representation shown in block diagram illustrating one embodiment of the invention in which both the condensate fraction and the wax fraction from the Fischer-Tropsch unit are passed to the hydrotreating zone.

FIG. 2 is a schematic representation of an alternate embodiment of the invention in which only the wax fraction from the Fischer-Tropsch unit is passed to the thermal cracking and oligomerization units.

FIG. 3 is a schematic representation of an embodiment of the invention in which the Fischer-Tropsch condensate fraction is passed to the thermal cracking and oligomerization units.

### DETAILED DESCRIPTION OF THE INVENTION

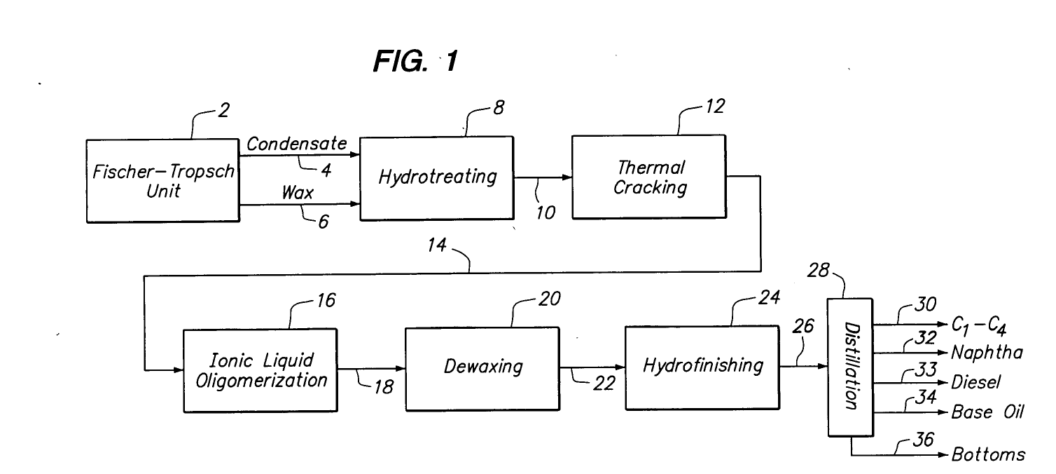

The present invention will be more clearly understood by referring to FIG. 1 which illustrates a simplified processing scheme showing the elements of the invention. Two separate Fischer-Tropsch feed streams are shown leaving the Fischer-Tropsch unit 2. They include a Fischer-Tropsch condensate feed 4 and a Fischer-Tropsch wax feed 6 shown as being carried to the hydrotreating unit 8 where the oxygenates and any nitrogen compounds in the feed streams are removed. In the hydrotreating unit, most of the unsaturated bonds in the hydrocarbon molecules are also saturated. The hydrotreated Fischer-Tropsch derived feed comprising a mixture of both the condensate and wax fractions is collected in line 10 and carried to the thermal cracking unit 12 where the paraffin molecules are cracked to form olefins. The olefin-enriched Fischer-Tropsch feed from the thermal cracker is carried by line 14 to the oligomerization unit 16 where the feed is contacted with an ionic liquid catalyst in order to increase the average molecular weight of the hydrocarbon molecules in the feed and introduce desirable branching into the molecule. The effluent from the oligomerization operation is carried by line 18 to the dewaxing unit 20 where the feed is dewaxed in order to further improve the flow properties of the product. The dewaxed product is sent via line 22 to a hydrofinishing unit 24 to saturate any remaining double bonds and improve the stability of the product. The hydrofinished product is sent by line 26 to the atmospheric and vacuum distillation unit 28 where the various products are separated. Shown in the figure exiting the distillation unit are the gaseous light products 30 which comprise the C₄ minus hydrocarbons, those hydrocarbons boiling within the range of naphtha 32, a diesel product 33, base oil products 34, and bottoms 36. The present scheme is intended to maximize the production of the higher value diesel and base oil products while minimizing the production of the gaseous light products. The bottoms fraction may be a heavy neutral base oil or bright stock or may be sent to another hydroprocessing unit for additional processing, if desired.

In the process scheme illustrated in FIG. 1, the condensate and wax fractions may be processed together (commingled). Alternately, the condensate and wax fractions may be processed in any of the steps in separate blocks in the same equipment or processed in separate reactors. The objective is to process each fraction at optimum conditions to maximize the yields and/or desirable properties of the desired products.

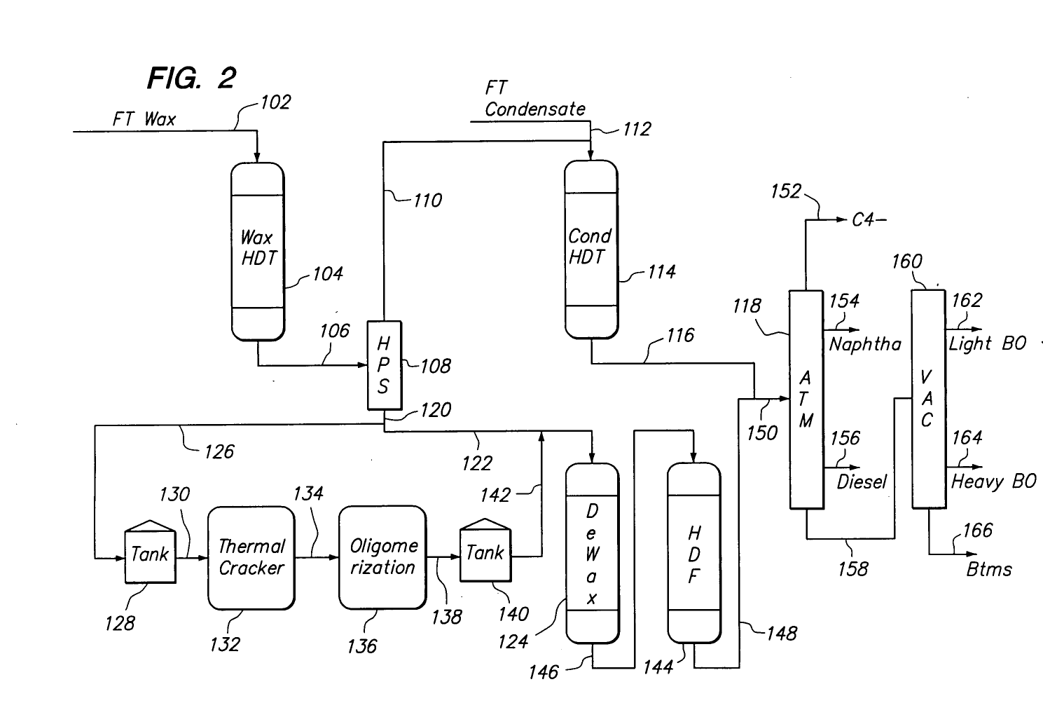

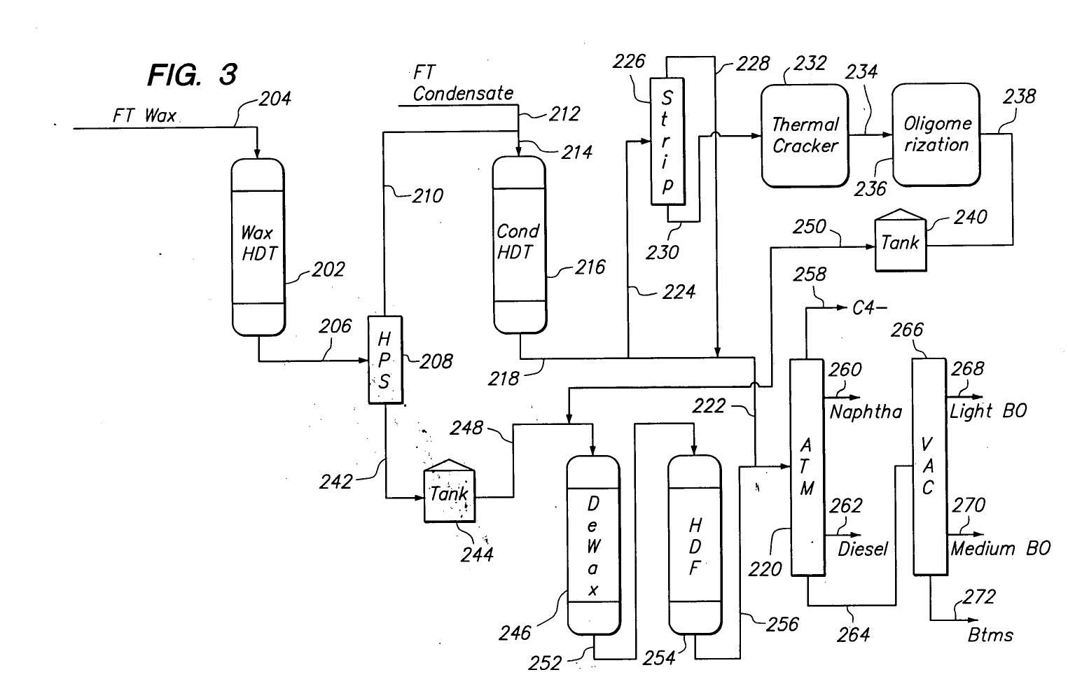

The process scheme illustrated in FIG. 1 shows both the condensate fraction and the wax fraction passing from the Fischer-Tropsch unit 2 to the hydrotreating, thermal cracking, and oligomerization units. Alternate embodiments of the invention may separate the condensate fraction and wax fraction and only one or the other of these fractions may pass to these units. For example, in order to minimize the amount of transportation fuel, especially naphtha, and maximize lubricant production, only the condensate fraction may pass to the hydrotreating, thermal cracking, and oligomerization units. In this embodiment, the wax fraction may pass directly to a dewaxing unit, with or without being hydrotreated first. In a different embodiment, only the wax fraction may be passed to the hydrotreating, thermal cracking, and oligomerization units with the condensate fraction passing directly to an atmospheric distillation unit, with or without first being hydrotreated, to collect the naphtha and diesel products. Alternate embodiments employing the invention in various processing schemes are further illustrated in FIGS. 2 and 3 which will further clarify how the invention may be used to produce different product slates, without limiting the scope of the invention.

FIG. 2 illustrates an alternate embodiment of the invention which is intended to produce lubricating base oil products having a high average molecular weight. The embodiment shown in FIG. 2 is suitable for the production of base oils from which a Fischer-Tropsch derived bright stock may be prepared. It is also suitable for the production of high yields of base oils having a higher viscosity than can be prepared by simply hydroisomerization dewaxing of Fischer-Tropsch wax. In this embodiment, the Fischer-Tropsch wax fraction and condensate fraction are recovered separately from the Fischer-Tropsch reactor (not shown). The Fischer-Tropsch wax fraction enters the wax hydrotreating unit 104 via feed line 102. In the wax hydrotreating unit, the amount of oxygenates and the nitrogen compounds present in the wax feed are reduced. The hydrotreated wax feed is carried by line 106 to a high pressure separator 108 where some lower boiling hydrocarbons, generally those boiling below about 650 degrees F. (about 345 degrees C.), are separated from the higher boiling base oil fractions. The hydrotreated lighter fraction comprising primarily hydrocarbons boiling within the range of transportation fuels, such as diesel and naphtha, is collected as overhead in line 110 and mixed with condensate carried from the Fischer-Tropsch reactor in line 112. The hydrotreated lighter fraction from the high pressure separator and the condensate fraction pass together to the condensate hydrotreating unit 114. The hydrotreated condensate fraction, which now includes the light fraction from the high pressure separator, is collected in line 116 which carries the condensate directly to the atmospheric distillation unit 118.

Returning to the high pressure separator, the higher boiling hydrotreated fraction is collected in line 120 and is divided into two hydrotreated heavy streams. One stream passes directly by line 122 to the dewaxing unit 124. The second stream passes by way of line 126 to a first storage tank 128 before passing to the thermal cracking and oligomerization operation. The amount of the hydrotreated heavy hydrocarbons sent to either the thermal cracking operation or the dewaxing unit will depend upon the amount of very heavy base oil product desired in the final product slate. The more heavy wax fraction that is sent to the thermal cracker, the more heavy-end lubricant base oils can be produced. The hydrotreated heavy oil fraction stored in first storage tank 128 is sent by line 130 to the thermal cracking unit 132 in which some of the paraffins are pyrolyzed to significantly increase the amount of olefins present in the feed. The olefin enriched heavy feed is sent via line 134 to the oligomerization unit 136 where the heavy feed is oligomerized in the presence of a Lewis acid ionic liquid catalyst. The oligomerized heavy feed is collected in line 138 and sent to a second storage tank 140.

The first and second storage tanks 128 and 140, respectively, allow the flexibility to operate the downstream dewaxing unit 124 in either block or bulk mode. In block mode, the dewaxing unit processes either hydrotreated Fischer-Tropsch wax (directly from the high pressure separator 108 by way of line 122) or oligomerization product (from storage second storage tank 140 by way of line 142). This mode allows the dewaxing conditions to be optimized for the specific feed to maximize dewaxing yield and product qualities. It also allows for the collection of separate base oils derived from oligomerization or dewaxing only.

In bulk dewaxing mode, the oligomerized product from line 142 and hydrotreated Fischer-Tropsch wax from line 122 are commingled and dewaxed together. The oligomerized heavy feed leaves the second storage tank by way of line 142 and is mixed with the heavy feed stream in line 122. This mixed heavy feed comprising both oligomerized heavy feed and heavy feed coming directly from the high pressure separator passes to the dewaxing unit 124.

The product from the dewaxing unit is sent to the hydrofinishing unit 144 by line 146. In the hydrofinishing unit, the base oils are stabilized and collected in line 148 where they are mixed with the condensate fraction in line 116. The combined heavy and condensate fractions are carried by line 150 to the atmospheric distillation unit 118 where the overhead gases 152 are separated from the naphtha 154 and the diesel 156. The bottoms from the atmospheric distillation unit is collected in line 158 and passed to a vacuum distillation unit 160 to separate the various base oil fractions. In the figure a light Fischer-Tropsch base oil product 162, a heavy Fischer-Tropsch base oil product 164, and a Fischer-Tropsch bottoms 166 are shown as being collected. The bottoms may be further refined to prepare bright stock if so desired and if necessary. If the bottoms product does not meet certain base oil specifications, such as pour point or cloud point, this stream may also be sent to the thermal cracking unit for further processing.

FIG. 3 illustrates a different embodiment of the invention in which the Fischer-Tropsch condensate is oligomerized using the process of the invention. In this embodiment, the Fischer-Tropsch wax fraction is carried to the wax hydrotreating unit 202 by line 204. The hydrotreated wax fraction is recovered in line 206 and sent to a high pressure separator 208 where the wax is separated from a lighter fraction as already described in the description of FIG. 2. The lighter fraction from the high pressure separator is collected by line 210 and mixed with the condensate fraction carried from the Fischer-Tropsch reactor in line 212. The mixture of condensate and light hydrocarbons from the high pressure separator are carried by common line 214 to the condensate hydrotreating reactor 216 where substantially all of the oxygenates and the nitrogen compounds are removed. The oxygenate-free condensate is collected by outlet line 218 and divided into two streams. One stream passes directly to the atmospheric distillation unit 220 via line 222. The second stream passes by way of line 224 to a stripper 226 where the C₄ minus hydrocarbons, ammonia, and water are removed. These overhead gases are collected by line 228 and sent to the atmospheric distillation unit 220. The condensate collected from the stripper passes by line 230 to the thermal cracker 232 where the paraffins are pyrolyzed to form olefins. The olefin-enriched condensate is carried via line 234 to the oligomerization unit 236. The oligomerized feed stream passes by way of line 238 to the condensate storage tank 240.

Returning to the high pressure separator 208, the hydrotreated heavy wax fraction is collected in line 242 and carried to hydrotreated wax storage tank 244. The condensate storage tank 240 and hydrotreated wax storage tank 244 allow the flexibility to operate the downstream dew-axing unit 246 in either block or bulk mode. In block mode, the dewaxing unit processes either hydrotreated Fischer-Tropsch wax (directly from the high pressure separator 208 by way of wax storage tank 244 and line 248) or oligomerization product from condensate storage tank 240 by way of line 250). In bulk dewaxing mode, the oligomerized product from line 250 and hydrotreated Fischer-Tropsch wax from line 248 are commingled and dewaxed together.

The product from the dewaxing unit 246 is sent by line 252 to the hydrofinishing unit 254 and from there passes by way of line 256 to the atmospheric distillation unit 220. In the atmospheric distillation unit, the overhead gases 258, naphtha 260, diesel 262 are separately collected. The bottoms from the atmospheric distillation unit is collected in line 264 and sent to the vacuum distillation unit 266 where light base oil 268, medium base oil 270, and bottoms 272 are shown as being separately collected.

The process scheme shown in FIG. 3 is very flexible. The source of the condensate may be either condensate that is collected directly from the Fischer-Tropsch plant or hydrocrackate that is recovered from a wax hydrocracker. In the process scheme illustrated in FIG. 3, the amount of base oils produced may be significantly increased as compared to the other process schemes described.

For clarity, the figures do not show hydrogen feed or recycle gas in the hydroprocessing units.

### Fischer-Tropsch Synthesis

During Fischer-Tropsch synthesis, liquid and gaseous hydrocarbons are formed by contacting a synthesis gas (syngas) comprising a mixture of hydrogen and carbon monoxide with a Fischer-Tropsch catalyst under suitable temperature and pressure reactive conditions. The Fischer-Tropsch reaction is typically conducted at temperatures of from about 300 degrees to about 700 degrees F. (about 150 degrees to about 370 degrees C.), preferably from about 400 degrees to about 550 degrees F. (about 205 degrees to about 290 degrees C.); pressures of from about 10 to about 600 psia (0.7 to 41 bars), preferably 30 to 300 psia (2 to 21 bars); and catalyst space velocities of from about 100 to about 10,000 cc/g/hr., preferably 300 to 3,000 cc/g/hr.

The products from the Fischer-Tropsch synthesis may range from C₁ to C₂₀₀ plus hydrocarbons with a majority in the C₅-C₁₀₀ plus range. The reaction can be conducted in a variety of reactor types, such as, for example, fixed bed reactors containing one or more catalyst beds, slurry reactors, fluidized bed reactors, or a combination of different types of reactors. Such reaction processes and reactors are well known and documented in the literature. The slurry Fischer-Tropsch process, which is preferred in the practice of the invention, utilizes superior heat (and mass) transfer characteristics for the strongly exothermic synthesis reaction and is able to produce relatively high molecular weight paraffinic hydrocarbons when using a cobalt catalyst. In the slurry process, a syngas comprising a mixture of hydrogen and carbon monoxide is bubbled up as a third phase through a slurry which comprises a particulate Fischer-Tropsch type hydrocarbon synthesis catalyst dispersed and suspended in a slurry liquid comprising hydrocarbon products of the synthesis reaction which are liquid under the reaction conditions. The mole ratio of the hydrogen to the carbon monoxide may broadly range from about 0.5 to about 4, but is more typically within the range of from about 0.7 to about 2.75 and preferably from about 0.7 to about 2.5. A particularly preferred Fischer-Tropsch process is taught in European Patent Application No. 0609079, which is completely incorporated herein by reference for all purposes.

Suitable Fischer-Tropsch catalysts comprise one or more Group VIII catalytic metals such as Fe, Ni, Co, Ru and Re, with cobalt being preferred. Additionally, a suitable catalyst may contain a promoter. Thus, a preferred Fischer-Tropsch catalyst comprises effective amounts of cobalt and one or more of Re, Ru, Pt, Fe, Ni, Th, Zr, Hf, U, Mg and La on a suitable inorganic support material, preferably one which comprises one or more refractory metal oxides. In general, the amount of cobalt present in the catalyst is between about 1 and about 50 weight percent of the total catalyst composition. The catalysts can also contain basic oxide promoters such as ThO₂, La₂O₃, MgO, and TiO₂, promoters such as ZrO₂, noble metals (Pt, Pd, Ru, Rh, Os, Ir), coinage metals (Cu, Ag, Au), and other transition metals such as Fe, Mn, Ni, and Re. Suitable support materials include alumina, silica, magnesia and titania or mixtures thereof. Preferred supports for cobalt containing catalysts comprise alumina or titania. Useful catalysts and their preparation are known and illustrated in U.S. Pat. No. 4,568,663, which is intended to be illustrative but non-limiting relative to catalyst selection.

The products as they are recovered from the Fischer-Tropsch operation usually may be divided into three fractions, a gaseous fraction consisting of very light products, a condensate fraction generally boiling in the range of naphtha and diesel, and a high boiling Fischer-Tropsch wax fraction which is normally solid at ambient temperatures.

### Hydrotreating to Remove the Oxygenates and Nitrogen

The wax and condensate recovered from the Fischer-Tropsch operation will contain varying amounts of oxygenates. Although the majority of the oxygenates are concentrated in the condensate, sufficient oxygenates may be present in the wax to interfere with the oligomerization operation when an ionic liquid catalyst is employed. The majority of the oxygenates are in the form of alcohols, however, lesser amounts of ketones, aldehydes, carboxylic acids, esters, and anhydrides may also be present. As already noted above, the presence of oxygenates in the feed to the oligomerization operation will result in the deactivation of the ionic liquid catalyst. Aside from the alcohols present, the most important oxygenates appear to be ketones and carboxylic acids.

In the present invention, the oxygenates present in the feed to the oligomerization operation, whether condensate or wax, are removed by hydrotreating. Hydrotreating also removes any nitrogen compounds which may present in the feed. The nitrogen content of the feed should be reduced to low levels (preferably less than 5 ppm) without excess cracking of the feedstock. “Hydrotreating” may be defined as a catalytic process, usually carried out in the presence of free hydrogen, in which the primary purpose when used to process conventional petroleum derived feed stocks is the removal of various contaminants, such as arsenic; heteroatoms, such as sulfur, oxygen, and nitrogen; and aromatics from the feed stock. In the present process, the primary purpose is to remove the oxygenates and nitrogen in the feed to the oligomerization operation. Generally, in hydrotreating operations, cracking of the hydrocarbon molecules, i.e., breaking the larger hydrocarbon molecules into smaller hydrocarbon molecules, is minimized. For the purpose of this discussion, the term “hydrotreating” refers to a hydroprocessing operation in which the cracking conversion is 20 percent or less.

Catalysts used in carrying out hydrotreating operations are well known in the art. See, for example, U.S. Pat. Nos. 4,347,121 and 4,810,357, the contents of which are hereby incorporated by reference in their entirety, for general descriptions of hydrotreating and of typical catalysts used in the process. Suitable catalysts include noble metals from Group VIIIA (according to the 1975 rules of the International Union of Pure and Applied Chemistry), such as platinum or palladium on an alumina or siliceous matrix, and Group VIIIA and Group VIB metals, such as nickel-molybdenum or nickel-tin on an alumina or siliceous matrix. In carrying out the present invention, hydrotreating catalysts containing the metals nickel and molybdenum are especially preferred. U.S. Pat. No. 3,852,207 describes a noble metal catalyst and mild conditions. Other suitable catalysts are described, for example, in U.S. Pat. Nos. 4,157,294 and 3,904,513. The non-noble hydrogenation metals, such as nickel-molybdenum, are usually present in the final catalyst composition as oxides, or more preferably or possibly, as sulfides when such compounds are readily formed from the particular metal involved. Preferred non-noble metal catalyst compositions contain in excess of about 5 weight percent, preferably about 5 to about 40 weight percent molybdenum and/or tungsten, and at least about 0.5, and generally about 1 to about 15 weight percent of nickel and/or cobalt determined as the corresponding oxides. Catalysts containing noble metals, such as platinum, contain in excess of 0.01 percent metal, preferably between 0.1 and 1.0 percent metal. Combinations of noble metals may also be used, such as mixtures of platinum and palladium.

The hydrogenation components can be incorporated into the overall catalyst composition by any one of numerous procedures. The hydrogenation components can be added to matrix component by co-mulling, impregnation, or ion exchange and the Group VI components, i.e., molybdenum and tungsten can be combined with the refractory oxide by impregnation, co-mulling or co-precipitation.

The matrix component or support can be of many types including some that have acidic catalytic activity; however, generally a non-acidic hydrotreating catalyst is preferred in carrying out the present invention, with alumina being especially preferred. Supports that have acidic activity include amorphous silica-alumina or may be a zeolitic or non-zeolitic crystalline molecular sieve. Examples of suitable matrix molecular sieves include zeolite Y, zeolite X and the so-called ultra stable zeolite Y and high structural silica:alumina ratio zeolite Y such as that described in U.S. Pat. Nos. 4,401,556; 4,820,402 and 5,059,567. Small crystal size zeolite Y, such as that described in U.S. Pat. No. 5,073,530, can also be used. Non-zeolitic molecular sieves which can be used include, for example, silicoaluminophosphates (SAPO), ferroaluminophosphate, titanium aluminophosphate and the various ELAPO molecular sieves described in U.S. Pat. No. 4,913,799 and the references cited therein. Details regarding the preparation of various non-zeolite molecular sieves can be found in U.S. Pat. Nos. 5,114,563 (SAPO) and 4,913,799 and the various references cited in U.S. Pat. No. 4,913,799. Mesoporous molecular sieves can also be used, for example, the M41S family of materials as described in J. Am. Chem. Soc., 114:10834-10843(1992), MCM-41; U.S. Pat. Nos. 5,246,689; 5,198,203 and 5,334,368; and MCM-48 (Kresge et al., Nature 359:710 (1992)). Suitable matrix materials may also include synthetic or natural substances as well as inorganic materials such as clay, silica and/or metal oxides such as silica-alumina, silica-magnesia, silica-zirconia, silica-thoria, silica-berylia, silica-titania as well as ternary compositions, such as silica-alumina-thoria, silica-alumina-zirconia, silica-alumina-magnesia, and silica-magnesia zirconia. The latter may be either naturally occurring or in the form of gelatinous precipitates or gels including mixtures of silica and metal oxides. Naturally occurring clays which can be composited with the catalyst include those of the montmorillonite and kaolin families. These clays can be used in the raw state as originally mined or initially subjected to calcination, acid treatment or chemical modification.

In performing the hydrotreating operation, more than one catalyst type may be used in the reactor. The different catalyst types can be separated into layers or mixed.

Typical hydrotreating conditions vary over a wide range. In general, the overall LHSV is usually between about 0.5 to 5.0, preferably between about 1.0 and 4.0. The total pressure ranging from about 200 psig to about 2,000 psig. Hydrogen recirculation rates are typically greater than 50 SCF/Bbl, and are preferably between 1,000 and 5,000 SCF/Bbl. Temperatures in the reactor will range from about 400 degrees F. to about 800 degrees F. (about 205 degrees C. to about 425 degrees C.), with temperatures of less than about 675 degrees F. (about 360 degrees C.) generally being preferred in the present process to avoid hydroisomerization.

In practicing the present invention, during the hydrotreating step, the amount of the~oxygenates are significantly reduced relative to the amount of oxygenates present in the Fischer-Tropsch derived feed entering the hydrotreating unit. As used herein, “significantly reduced” means that the elemental oxygen remaining in the hydrotreated feed is about 1500 ppmw or less. Preferably, substantially all of oxygenates are removed in the hydrotreating step. Using the present invention, the effluent from the hydrotreating operation preferably will contain less than about 200 ppmw elemental oxygen, even more preferably less than 100 ppmw elemental oxygen. However, while it is relatively easy to achieve these levels by hydrotreating the condensate, it has been found that more severe hydrotreating conditions may be required to reach these levels when the wax fraction is being treated. Consequently, as a practical matter, it may be desirable to allow oxygenate levels in excess of these preferred amounts to remain in the wax fraction and accept some deactivation of the ionic liquid oligomerization catalyst. The deactivation of the ionic liquid catalyst in this later instance requires that additional make-up catalyst be added to the oligomerization zone to replace the catalyst deactivated by the residual oxygenates.

### Thermal Cracking

The thermal cracking step employed in the process of the present invention is intended to crack the paraffin molecules in the Fischer-Tropsch feed into lower molecular weight olefins. Although Fischer-Tropsch wax and condensate usually contain a significant amount of olefins, in the present invention, most of the olefins are saturated in the hydrotreating operation. Therefore, it is necessary to reintroduce sufficient olefins into the feed to allow for the oligomerization step to proceed.

Although batch pyrolysis reactors such as employed in delayed coking or in cyclic batch operations could be used to carry out this step, generally a continuous flow-through operation is preferred in which the Fischer-Tropsch feed is first preheated to a temperature sufficient to vaporize most or all of the feed after which the vapor is passed through a tube or tubes. The conditions in the flow through reactor are critical to the optimal formation of olefins from the paraffins present in the substantially oxygenate-free Fischer-Tropsch derived feed. The temperature of the feed must be raised to a temperature sufficient to vaporize most or all of the feed. A desirable option is to bleed any remaining nonvaporized hydrocarbons prior to entering the cracking furnace. Liquid cracking has been found to lead to the formation of undesired paraffins. Preferably, the thermal cracking is conducted in the presence of steam which serves as a heat source and also helps suppress coking in the reactor. Details of a typical steam thermal cracking process may be found in U.S. Pat. No. 4,042,488, hereby incorporated by reference in its entirety. Although catalyst is generally not used in carrying out the thermal cracking operation, it is possible to conduct the operation in a fluidized bed in which the vaporized feed is contacted with hot fluidized inert particles, such as fluidized particles of coke.

In performing the thermal cracking operation, it is preferable that the feed be maintained in the vapor phase during the cracking operation to maximize the production of olefins. In the thermal cracking zone, the cracking conditions should be sufficient to provide a cracking conversion of greater than 10 weight percent of the paraffins present. The optimal temperature and other conditions in the thermal cracking zone for the cracking operation will vary somewhat depending on the feed. In general, the temperature must be high enough to maintain the feed in the vapor phase but not so high that the feed is overcracked, i.e., the temperature and conditions should not be so severe that excessive C₄ minus hydrocarbons are generated. The temperature in the thermal cracking zone normally will be maintained at a temperature of between about 950 degrees F. (510 degrees C.) and about 1,600 degrees F. (870 degrees C.). The optimal temperature range for the thermal cracking zone in order to maximize the production of olefins from the Fischer-Tropsch feed will depend upon the endpoint of the feed. In general, the higher the carbon number, the higher the temperature required to achieve maximum conversion. Accordingly, some routine experimentation may be necessary to identify the optimal cracking conditions for a specific feed. The thermal cracking zone usually will employ pressures maintained between about 0 atmospheres and about 5 atmospheres, with pressures in the range of from about 0 to about 2 generally being preferred. Although the optimal residence time of the feed in the reactor will vary depending on the temperature and pressure in the thermal cracking zone, typical residence times are generally in the range of from about 1.5 seconds to about 500 seconds, with the preferred range being between about 5 seconds and about 300 seconds.

### Oligomerization

Following pyrolysis, the olefin-enriched Fischer-Tropsch feed is oligomerized using a Lewis acid ionic liquid catalyst. The use of an ionic liquid catalyst for the oligomerization of the olefins in the present invention has certain advantages over more conventional catalysts in that there is excellent mixing of the reactants with the catalyst resulting in short residence times and high yields, the oligomerization reaction takes place at relatively low temperatures, and the products are readily separated from the catalyst. In the present process, the olefin-enriched Fischer-Tropsch feed may be added to the catalytic mixture or the catalyst may be added to the feed. In either case, the feed and the product formed during oligomerization will form a separate phase from the ionic liquid which allows the two phases to be readily separated. In order to facilitate mixing of the ionic liquid catalyst and the feed, it is desirable to either stir the oligomerization mixture, bubble the feed through the ionic liquid catalyst, or use another type of reactor which facilitates good mixing of the catalyst and the hydrocarbon. Following completion of the oligomerization reaction, the mixing should be halted, and the product and residual feed should be allowed to form a distinct layer apart from the catalyst phase.

The ionic liquid oligomerization catalyst used in this invention will be a Lewis acid catalyst and usually will comprise at least two components which form a complex. In most instances, the catalyst will be a binary catalyst, i.e., it will consist of only two components. The first component of the catalyst will usually comprise a Lewis acid selected from the group consisting of aluminum halide, alkyl aluminum halide, gallium halide, and alkyl gallium halide. Preferred for the first component is an aluminum halide or alkyl aluminum halide. Aluminum trichloride is particularly preferred for preparing the oligomerization catalyst used in practicing the present invention. The presence of the first component should give the ionic liquid a Lewis (or Franklin) acidic character.

The second component making up the catalyst is usually a quaternary ammonium or quaternary phosphonium compound, such as, for example, a salt selected from one or more of hydrocarbyl substituted ammonium halides, hydrocarbyl substituted imidizolium halide, hydrocarbyl substituted pyridinium halide, alkylene substituted pyridinium dihalide, hydrocarbyl substituted phosphonium halide. Preferred for use as the second component are those quaternary ammonium halides containing one or more alkyl moieties having from 1 to about 9 carbon atoms, such as, for example, trimethylamine hydrochloride, methyl-tributyl ammonium chloride, or alkyl substituted imidazolium halides, such as, for example, 1-ethyl-3-methyl-imidazolium chloride.

The mole ratio of the two components will usually fall within the range of from about 1:1 to about 5:1 of said first component to said second component, and more preferably the mole ratio will be in the range of from about 1:1 to about 2:1. The use of a binary catalyst composition consisting essentially of methyl-tributyl ammonium chloride and aluminum trichloride is particularly advantageous for carrying out the process of the present invention due to the ease of preparation, the ready commercial availability of the components, and the relatively low cost.

The amount of catalyst present to promote the oligomerization of the olefins should be not less than an effective oligomerizing amount, that is to say, the minimum amount of the catalyst necessary to olgomerize the olefins to the desired product. This may vary to some degree depending on the composition of the catalyst, the ratio of the two components of the catalyst to one another, the feed, the oligomerzation conditions chosen, and the like. However, a determination of the effective catalytic amount should be well within the ability of one skilled in the art with no more than routine testing necessary to establish the amount needed to carry out the invention-As noted above, make-up catalyst added to the oligomerization zone may be necessary to replace catalyst that is deactivated by contaminants in the feed, mostly residual oxygenates present in the wax fraction. The amount of make-up catalyst necessary will depend on the amount of contaminants present. Preferably, the amount of contaminants will be low and the degree of deactivation of the catalyst also will be low. However, if the removal of the oxygenates to the most preferred levels during the hydrotreating step require operation at such high severity that significant cracking takes place and the amount of desirable high molecular weight products are correspondingly reduced, it may be necessary to tolerate some catalyst deactivation in order to produce the desired product slate.

The oligomerization reaction takes place over a wide temperature range between the melting point of the catalyst and its decomposition temperature, preferably between about 120 degrees F. and about 212 degrees F. (about 50 degrees C. and about 100 degrees C.).

Following completion of the oligomerization reaction, the organic layer containing the Fischer-Tropsch derived oligomerization product is separated from the ionic liquid phase. Preferably, the oligomerization product will have an average molecular weight at least 10 percent higher than the initial olefin-enriched Fischer-Tropsch feedstock, more preferably at least 20 percent higher. The acidic ionic liquid catalyst that remains after recovery of the organic phase is preferably recycled to the oligomerization zone.

### Dewaxing

The product from the oligomerization unit may require dewaxing to meet the lubricant base oil cold flow requirements. The dewaxing process may be a solvent or a catalytic process. Catalytic dewaxing is generally preferred, especially for the process schemes where some of the Fischer-Tropsch wax is hydroisomerized to lubricant base oil. In these schemes the catalytic dewaxing unit can operate in either of two modes, (1) feeding the oligomerization product or (2) feeding Fischer-Tropsch wax.

Catalytic dewaxing consists of three main classes, conventional hydrodewaxing, complete hydroisomerization dewaxing, and partial hydroisomerization dewaxing. All three classes involve passing a mixture of a waxy hydrocarbon stream and hydrogen over a catalyst that contains an acidic component to convert the normal and slightly branched iso-paraffins in the feed to other non-waxy species, such as lubricating base oil stocks with acceptable pour points. Typical conditions for all classes involve temperatures from about 400 degrees F. to about 800 degrees F. (about 200 degrees C. to about 425 degrees C.), pressures from about 200 psig to 3,000 psig, and space velocities from about 0.2 to 5 hr⁻¹. The method selected for dewaxing a feed typically depends on the product quality, and the wax content of the feed, with conventional hydrodewaxing often preferred for low wax content feeds. The method for dewaxing can be effected by the choice of the catalyst. The general subject is reviewed by Avilino Sequeira, in Lubricant Base Stock and Wax Processing, Marcel Dekker, Inc., pages 194-223. The determination between conventional hydrodewaxing, complete hydroisomerization dewaxing, and partial hydroisomerization dewaxing can be made by using the n-hexadecane isomerization test as described in U.S. Pat. No. 5,282,958. When measured at 96 percent, n-hexadecane conversion using conventional hydrodewaxing catalysts will exhibit a selectivity to isomerized hexadecanes of less than 10 percent, partial hydroisomerization dewaxing catalysts will exhibit a selectivity to isomerized hexadecanes of greater than 10 percent to less than 40 percent, and complete hydroisomerization dewaxing catalysts will exhibit a selectivity to isomerized hexadecanes of greater than or equal to 40 percent, preferably greater than 60 percent, and most preferably greater than 80 percent.

In conventional hydrodewaxing, the pour point is lowered by selectively cracking the wax molecules mostly to smaller paraffins using a conventional hydrodewaxing catalyst, such as, for example, ZSM-5. Metals may be added to the catalyst, primarily to reduce fouling.

Complete hydroisomerization dewaxing typically achieves high conversion levels of wax by isomerization to non-waxy iso-paraffins while at the same time minimizing the conversion by cracking. Since wax conversion can be complete, or at least very high, this process typically does not need to be combined with additional dewaxing processes to produce a lubricating base oil stock with an-acceptable pour point. Complete hydroisomerization dewaxing uses a dual-functional catalyst consisting of an acidic component and an active metal component having hydrogenation activity. Both components are required to conduct the isomerization reaction. The acidic component of the catalysts used in complete hydroisomerization preferably includes an intermediate pore SAPO, such as SAPO-11, SAPO-31, and SAPO-41, with SAPO-11 being particularly preferred. Intermediate pore zeolites, such as ZSM-22, ZSM-23, and SSZ-32, also may be used in carrying out complete hydroisomerization dewaxing. Typical active metals include molybdenum, nickel, vanadium, cobalt, tungsten, zinc, platinum, and palladium. The metals platinum and palladium are especially preferred as the active metals, with platinum most commonly used.

In partial hydroisomerization dewaxing, a portion of the wax is isomerized to iso-paraffins using catalysts that can isomerize paraffins selectively, but only if the conversion of wax is kept to relatively low values (typically below 50 percent). At higher conversions, wax conversion by cracking becomes significant, and yield losses of lubricating base oil stock become uneconomical. Like complete hydroisomerization dewaxing, the catalysts used in partial hydroisomerization dewaxing include both an acidic component and a hydrogenation component. The acidic catalyst components useful for partial hydroisomerization dewaxing include amorphous silica aluminas, fluorided alumina, and 12-ring zeolites (such as Beta, Y zeolite, L zeolite). The hydrogenation component of the catalyst is the same as already discussed with complete hydroisomerization dewaxing. Because the wax conversion is incomplete, partial hydroisomerization dewaxing must be supplemented with an additional dewaxing technique, typically solvent dewaxing, complete hydroisomerization dewaxing, or conventional hydrodewaxing in order to produce a lubricating base oil stock with an acceptable pour point.

In preparing those catalysts containing a SAPO non-zeolitic molecular sieve and having a hydrogenation component for use in the present invention, it is usually preferred that the metal be deposited on the catalyst using a non-aqueous method. Catalysts containing SAPOs on which the metal has been deposited using a non-aqueous method have shown greater selectivity and activity than those catalysts which have used an aqueous method to deposit the active metal. The non-aqueous deposition of active metals on non-zeolitic molecular sieves is taught in U.S. Pat. No. 5,939,349. In general, the process involves dissolving a compound of the active metal in a non-aqueous, non-reactive solvent and depositing it on the molecular sieve by ion exchange or impregnation.

For the purposes of the present invention, hydroisomerization dewaxing, especially complete hydroisomerization dewaxing, is preferred over hydrodewaxing if such operation is able to provide the desired viscosity and pour point specifications for the product. This is because with less wax cracking, the yield of lubricating base oil will be increased. The preferred hydroisomerization catalyst for use in the catalytic hydroisomerization step comprises SAPO-11.

### Hydrofinishing

Hydrofinishing operations are intended to improve the UV stability and color of the Fischer-Tropsch derived products recovered from the oligomerization zone. It is believed this is accomplished by saturating the double bonds present in the hydrocarbon molecule. A general description of the hydrofinishing process may be found in U.S. Pat. Nos. 3,852,207 and 4,673,487. As used in this disclosure, the term “UV stability” refers to the stability of the lubricating base oil or other products when exposed to ultraviolet light and oxygen. Instability is indicated when a visible precipitate forms or darker color develops upon exposure to ultraviolet light and air which results in a cloudiness or floc in the product. Lubricating base oils and diesel products prepared by-the process of the present invention will require UV stabilization before they are suitable for use in the manufacture of commercial lubricating oils and marketable diesel.

In the present invention, the total pressure in the hydrofinishing zone will be above 500 psig and preferably above 1,000 psig. The maximum total pressure is not critical to the process; but due to equipment limitations, the total pressure will not exceed 3,000 psig and usually will not exceed about 2,500 psig. Temperature ranges in the hydrofinishing zone are usually in the range of from about 300 degrees F. (150 degrees C.) to about 700 degrees F. (370 degrees C.), with temperatures of from about 400 degrees F. (205 degrees C.) to about 500 degrees F. (260 degrees C.) being preferred. The LHSV is usually within the range of from about 0.2 to about 2.0, preferably 0.2 to 1.5, and most preferably from about 0.7 to 1.0. Hydrogen is usually supplied to the hydrofinishing reactor at a rate of from about 1,000 to about 10,000 SCF per barrel of feed. Typically, the hydrogen is fed at a rate of about 3,000 SCF per barrel of feed.

Suitable hydrofinishing catalysts typically contain a Group VII noble metal component together with an oxide support. Metals or compounds of the following metals are contemplated as useful in hydrofinishing catalysts include ruthenium, rhodium, iridium, palladium, platinum, and osmium. Preferably, the metal or metals will be platinum, palladium or mixtures of platinum and palladium. The refractory oxide support usually consists of silica-alumina, silica-alumina-zirconia, and the like. Typical hydrofinishing catalysts are disclosed in U.S. Pat. Nos. 3,852,207; 4,157,294 and 4,673,487.

### Distillation

The separation of the Fischer-Tropsch derived products into the various fractions is generally conducted by either atmospheric or vacuum distillation or by a combination of atmospheric and vacuum distillation. Atmospheric distillation is typically used to separate the lighter distillate fractions, such as naphtha and middle distillates, from a bottoms fraction having an initial boiling point above about 650 degrees F. to about 750 degrees F. (about 340 degrees C. to about 400 degrees C.). At higher temperatures, thermal cracking of the hydrocarbons may take place leading to fouling of the equipment and to lower yields of the heavier cuts. Vacuum distillation is typically used to separate the higher boiling material, such as the lubricating base oil fractions.

As used in this disclosure, the term “distillate fraction” or “distillate” refers to a side stream product recovered either from an atmospheric fractionation column or from a vacuum column as opposed to the “bottom fraction” which represents the residual higher boiling fraction recovered from the bottom of the column. In this disclosure, the term “bottoms” also includes those bottoms fractions and bright stock derived from the oligomerization of olefins present in the Fischer-Tropsch feed streams.

What is claimed is:

1. A process for oligomerizing a Fischer-Tropsch derived feed containing oxygenates which comprises:
(a) reducing significantly the oxygenates present in the Fischer-Tropsch derived feed by contacting said feed with a hydrotreating catalyst under hydrotreating conditions in a hydrotreating zone and recovering from the hydrotreating zone a Fischer-Tropsch derived hydrotreated feed which contains a significantly reduced amount of oxygenates as compared to the Fischer-Tropsch derived feed and also a significant amount of paraffins;
(b) pyrolyzing the Fischer-Tropsch derived hydrotreated feed in a thermal cracking zone under thermal cracking conditions pre-selected to crack the paraffin molecules to form olefins and collecting an olefin-enriched Fischer-Tropsch feed from the thermal cracking zone;
(c) contacting the olefin-enriched Fischer-Tropsch feed with a Lewis acid ionic liquid catalyst in an oligomerization zone under oligomerization reaction conditions; and p1 (d) recovering from the oligomerization zone a Fischer-Tropsch derived product having molecules characterized by a higher average molecular weight and increased branching as compared to the Fischer-Tropsch derived feed.

2. The process of claim 1 wherein the Fischer-Tropsch derived hydrotreated feed is substantially free of oxygenates.

3. The process of claim 2 wherein the Fischer-Tropsch derived hydrotreated feed contains less than 200 ppmw elemental oxygen.

4. The process of claim 3 wherein the Fischer-Tropsch derived hydrotreated feed contains less than 100 ppmw elemental oxygen.

5. The process of claim 1 wherein the hydrotreating catalyst is a non-acidic hydrotreating catalyst.

6. The process of claim 5 wherein the hydrotreating catalyst contains the metal nickel and molybdenum.

7. The process of claim 1 wherein the hydrotreating conditions in the hydrotreating zone include a temperature of between about 400 degrees F. and about 800 degrees F., an LHSV of between about 0.5 and about 5.0, and a total pressure between about 200 psig and about 2,000 psig.

8. The process of claim 7 wherein the temperature in the hydrotreating zone is less than about 675 degrees F.

9. The process of claim 7 wherein the LHSV is between about 1 and about 4.0.

10. The process of claim 1 wherein the temperature in the thermal cracking zone is within the range of from about 950 degrees F. and about 1,600 degrees F.

11. The process of claim 1 wherein the pressure in the thermal cracking zone is within the range of from about to about 0 atmospheres and about 5 atmospheres.

12. The process of claim 11 wherein the pressure in the thermal cracking zone is within the range of from about to about 0 atmospheres and about 2 atmospheres.

13. The process of claim 1 wherein the cracking conversion in the thermal cracking zone is greater than about 10 weight percent of the paraffins present.

14. The process of claim 1 wherein the ionic liquid oligomerization catalyst comprises a first component and a second component, said first component comprising a compound selected from the group consisting of aluminum halide, alkyl aluminum halide, gallium halide, and alkyl gallium halide, and said second component is a quaternary ammonium, or quaternary phosphonium salt.

15. The process of claim 14 wherein the ratio of the first component to the second component is within the range of from about 1:1 to about 2:1.

16. The process of claim 14 wherein said first component is aluminum halide or alkyl aluminum halide.

17. The process of claim 14 wherein said second component is selected from one or more of hydrocarbyl substituted ammonium halide, hydrocarbyl substituted imidazolium halide, hydrocarbyl substituted pyridinium halide, alkylene substituted pyridinium dihalide, or hydrocarbyl substituted phosphonium halide.

18. The process of claim 1 including the additional step of dewaxing the Fischer-Tropsch derived product recovered from the oligomerization zone and collecting a dewaxed Fischer-Tropsch product having improved cold flow properties relative to the Fischer-Tropsch derived product recovered from the oligomerization zone.

19. The process of claim 18 wherein the Fischer-Tropsch derived product is catalytically dewaxed.

20. The process of claim 18 including the additional step of hydrofinishing the dewaxed Fischer-Tropsch product.

21. The process of claim 1 wherein the Fischer-Tropsch derived product includes lubricant base oil.

22. The process of claim 1 wherein the Fischer-Tropsch derived product includes a diesel product.

23. A process for producing Fischer-Tropsch derived lubricant base oil which comprises:
(a) recovering from a Fischer-Tropsch plant a wax fraction;
(b) reducing significantly the oxygenates present in the Fischer-Tropsch wax fraction by contacting said wax fraction with a hydrotreating catalyst under hydrotreating conditions in a hydrotreating zone and recovering from the hydrotreating zone a hydrotreated Fischer-Tropsch derived wax feed which contains a significantly reduced amount of oxygenates as compared to the Fischer-Tropsch derived wax fraction and also a significant amount of paraffins;
(c) pyrolyzing the hydrotreated Fischer-Tropsch derived wax feed in a thermal cracking zone under thermal cracking conditions pre-selected to crack the paraffin molecules to form olefins and collecting an olefin-enriched Fischer-Tropsch feed from the thermal cracking zone;
(d) contacting the olefin-enriched Fischer-Tropsch feed with a Lewis acid ionic liquid catalyst in an oligomerization zone under oligomerization reaction conditions;
(e) recovering from the oligomerization zone a Fischer-Tropsch derived oligomerization effluent having molecules characterized by a higher average molecular weight and increased branching as compared to the Fischer-Tropsch derived feed;
(f) catalytically dewaxing the Fischer-Tropsch derived oligomerization effluent by contacting the Fischer-Tropsch derived oligomerization effluent with a dewaxing catalyst under catalytic conditions in a dewaxing zone and collecting a dewaxed Fischer-Tropsch product from the dewaxing zone having improved cold flow properties relative to the Fischer-Tropsch derived oligomerization effluent;
(g) hydrofinishing the dewaxed Fischer-Tropsch product in a hydrofinishing zone under hydrofinishing conditions in the presence of a hydrofinishing catalyst; and
(h) collecting a Fischer-Tropsch derived lubricant base oil from the hydrofinishing zone.

24. The process of claim 23 wherein the oxygenates in the hydrotreated Fischer-Tropsch derived wax feed recovered from the hydrotreating zone is substantially oxygenate free.

25. The process of claim 24 wherein the hydrotreated Fischer-Tropsch derived wax feed recovered from the hydrotreating zone contains less than 200 ppmw elemental oxygen.

26. A process for producing Fischer-Tropsch derived lubricant base oil which comprises:
(a) recovering from a Fischer-Tropsch plant a condensate fraction;
(b) removing substantially all of the oxygenates present in the Fischer-Tropsch condensate fraction by contacting said condensate fraction with a hydrotreating catalyst under hydrotreating conditions in a hydrotreating zone and recovering from the hydrotreating zone a substantially oxygenate-free Fischer-Tropsch derived condensate feed which also contains a significant amount of paraffins;
(c) pyrolyzing the substantially oxygenate-free Fischer-Tropsch derived condensate feed in a thermal cracking zone under thermal cracking conditions pre-selected to crack the paraffin molecules to form olefins and collecting an olefin-enriched Fischer-Tropsch feed from the thermal cracking zone;
(d) contacting the olefin-enriched Fischer-Tropsch feed with a Lewis acid ionic liquid catalyst in an oligomerization zone under oligomerization reaction conditions;
(e) recovering from the oligomerization zone a Fischer-Tropsch derived oligomerization effluent having molecules characterized by a higher average molecular weight and increased branching as compared to the Fischer-Tropsch derived feed;
(f) catalytically dewaxing the Fischer-Tropsch derived oligomerization effluent by contacting the Fischer-Tropsch derived oligomerization effluent with a dewaxing catalyst under catalytic conditions in a dewaxing zone and collecting a dewaxed Fischer-Tropsch product from the dewaxing zone having improved cold flow properties relative to the Fischer-Tropsch derived oligomerization effluent;
(g) hydrofinishing the dewaxed Fischer-Tropsch product in a hydrofinishing zone under hydrofinishing conditions in the presence of a hydrofinishing catalyst; and
(h) collecting a Fischer-Tropsch derived lubricant base oil from the hydrofinishing zone.

27. The process of claim 26 wherein the substantially oxygenate-free Fischer-Tropsch derived condensate feed recovered from the hydrotreating zone contains less than 200 ppmw elemental oxygen.

28. The process of claim 27 wherein the substantially oxygenate-free Fischer-Tropsch derived condensate feed recovered from the hydrotreating zone contains less than 100 ppmw elemental oxygen.

29. The process of claim 26 wherein a diesel product is also collected from the hydrofinishing zone.
